(19) United States
(12) Patent Application Publication
(10) Pub. No.: US 2023/0108715 A1
(43) Pub. Date: Apr. 6, 2023

(54) METHOD AND SYSTEM FOR THE NON-INVASIVE RECORDING OF MARINE MAMMAL SLEEP IN THE WILD

(71) Applicant: The Regents of the University of California, Oakland, CA (US)

(72) Inventors: Jessica Kendall-Bar, Santa Cruz, CA (US); Terrie Williams, Santa Cruz, CA (US); Patricio Guerrero, Santa Cruz, CA (US); Ethan Slattery, Santa Cruz, CA (US); Daniel P. Costa, Santa Cruz, CA (US)

(73) Assignee: The Regents of the University of California, Oakland, CA (US)

(21) Appl. No.: 17/959,146

(22) Filed: Oct. 3, 2022

Related U.S. Application Data
(60) Provisional application No. 63/251,532, filed on Oct. 1, 2021.

(51) Int. Cl. A61B 5/00; A61B 5/367; A61B 5/333

(52) U.S. Cl. CPC A61B 5/4806; A61B 5/367; A61B 5/333; A61B 2562/225; A61B 2503/40; A61B 2562/222; A61B 2562/182

(57) ABSTRACT

A water resistant biometric data logging system for an animal including a housing containing an electrophysiological data logging device and an underwater connector configured to route and waterproof at least 10 electrode cables to the electrophysiological data logging device. Further disclosed is an apparatus (e.g., headcap) for mounting electrodes onto the animal. The apparatus includes a first layer comprising a first plurality of openings or holes, the first layer comprising a foam or a sacrificial material; a second layer comprising a second plurality of openings or holes, the second layer comprising or consisting essentially of synthetic rubber; and a potted piece containing a plurality of electrode cables, wherein the electrode cables are routed from the potted piece through the first plurality of openings and the second plurality of openings or holes.

### CROSS REFERENCE TO RELATED APPLICATIONS

This application claims the benefit under 35 U.S.C. Section 119(e) of co-pending and commonly-assigned U.S. Provisional Patent Application Ser. No. 63/251,532, filed on Oct. 1, 2021, by Jessica M. Kendall-Bar, Daniel P. Costa, Patricio Guerrero, Ethan Slattery, and Terrie M. Williams, entitled “METHOD AND SYSTEM FOR THE NON-INVASIVE RECORDING OF MARINE MAMMAL SLEEP IN THE WILD”, (284.0007-US-P1); which application is incorporated by reference herein.

### STATEMENT REGARDING FEDERALLY SPONSORED RESEARCH AND DEVELOPMENT

This invention was made with Government support under Grant No. N00014-19-1-2178, awarded by the ONR. The Government has certain rights in the invention

### BACKGROUND OF THE INVENTION

### 1. Field of the Invention

The present invention relates to methods and systems for collecting biometric data.

### 2. Description of the Related Art

Hans Berger performed the first non-invasive scalp electroencephalogram (EEG) recording on a human shortly after the first invasive EEG recording in 1924 (Berger, 1929; İnce et al., 2020). Although the use of surface recordings for human clinical EEG applications is widespread, the majority of EEG studies on animals still involve electrode implantation (de Camp et al., 2018; Lyamin et al., 2012; Rattenborg et al., 2008; Scriba et al., 2013; Ternman et al., 2012). Only a handful of animal sleep studies have employed non-invasive EEG methods, including in owls, rats, and domestic animals such as dairy cows and piglets (Cousillas et al., 2017; de Camp et al., 2018; Paulson et al., 2018; Scriba et al., 2013; Ternman et al., 2012).

Almost all previous EEG studies on marine mammals rely on drilling holes into the skull as illustrated in FIG. 1 (Lyamin, 1993; Lyamin, Mukhametov, et al., 2002; Lyamin, Manger, et al., 2008; Lyamin et al., 2012; Lyamin & Chetyrbok, 1992; Lyamin & Siegel, 2019; Mukhametov et al., 1985; Ridgway et al., 1975; Ridgway, 2002). A very few marine mammal sleep studies have employed minimally invasive needle electrodes (Castellini et al., 1994; Milsom et al., 1996; Serafetidines et al., 1972), and these authors often reported difficulty keeping the needles in place over time (lasted<12 hours: (Lyamin, Manger, et al., 2008), M Harris personal communication). Other studies employed “harpoon-like” needle electrodes for recording EMG signals, which had barbs to keep the sensors in place (Lyamin, Shpak, et al., 2002; Mukhametov et al., 1976). What is needed are less invasive methods of electrophysiological monitoring of animals in the wild. The present disclosure satisfies this need.

### SUMMARY

Disclosed herein is a system comprising non-invasive surface mounted electrodes to monitor sleep in marine mammals. Said system is configured to monitor sleep in the wild: both on land (e.g. for pinnipeds) and in water, including at depth.

The system further includes a pressure proof recording device.

The system further includes methods of signal processing that maximize the quality of data collected the system.

It is an object of the system to maximize the animal's freedom of movement. A device according to embodiments described herein can be embodied in many ways including, but not limited to, the following.

1. A water resistant biometric data logging system for an animal, the system comprising (e.g., see FIG. 3):
(a) an electrophysiological data logging device;
(b) an underwater connector configured to route signals to the electrophysiological data logging device;
(c) a housing containing the electrophysiological data logging device, wherein:
   the housing is configured to accept a plurality of electrode cables connected to the underwater connector, and
   the housing comprises an aluminum wall (lightweight and strong for pressure, high density polycarbonate glass failed) surrounding the electrophysiological data logging device and a first acrylic window within the first aluminum wall configured to allow visualization of the electrophysiological data logging device; and
a flexible nylon mesh attached to the housing and configured to affix the housing to an outside of the animal.

2. The system of example 1, further comprising a plug comprising a screw configured to be screwed into a screw hole in the housing, wherein the plug includes an opening receiving the underwater connector so as to operably connect the underwater connector to the housing and the data logging device.

3. The system of example 1, wherein:
the system further comprises zip ties attaching the housing the mesh and epoxy for gluing the mesh to the animal, and
the housing comprises a cylinder or a capsule.

4. The system of example 1 wherein the electrode cables are soldered to the underwater connector, thereby forming a plurality solder joints, and wherein each of the solder joints are potted into a flexible epoxy splice joint.

5. The system of any of examples 1 or 4, wherein the connector comprises more than 10 pin connectors or at least 21 pin connectors, wherein each of the pin connectors comprises a solder joint soldered to a different one of the electrode cables and each of the solder joints is potted in the underwater connector for waterproofing.

6. The system of examples 4 or 5 wherein each of the solder joints is covered in marine grade heat-shrink.

7. The system of any of the examples 1-6, wherein each of the electrode cables comprises aerospace grade electrical shielding with 3/16 stainless steel around a wire.

8. The system of any of the examples 1-7 comprising at least 5, 10, 15, 20, 21, or 25 of the electrode cables.

9. The system of example 1 wherein one or more of the electrode cables terminate in a headcap, the headcap comprising a first neoprene layer, a mold configured to accept the plurality of electrode cables beneath the first neoprene layer; and a second neoprene layer between the mold and the first neoprene layer configured to hold the electrodes in place against the skin of the animal.

10. The system of example 1 wherein at least one of the plurality of electrode cables comprises electrical shielding around the at least one of the electrode cables.

11. The system of example 10 where the electrical shielding comprises braided copper or aluminum foil.

12. FIG. 2 illustrates an apparatus for mounting electrodes onto a body (e.g., of an animal or human), comprising:
a first layer comprising a first plurality of openings or holes, the first layer comprising or consisting essentially of synthetic rubber;
a second layer comprising a second plurality of openings or holes, the second layer comprising a foam or a sacrificial material;
a potted piece containing a plurality of electrode cables, wherein:
   the electrode cables are routed through the first plurality of openings and the second plurality of openings or holes, and
   the first layer is between the potted piece and the second layer;
a plurality of electrodes terminating the cables, wherein the electrodes are configured to be mounted (e.g., adhered to) a surface of the body; and
an adhesive between the first layer and the second layer, wherein the adhesive adheres the electrode cables to the second layer.

13. The apparatus of example 12, wherein the first layer and the second layer comprise or consist essentially of neoprene.

14. The apparatus of example 13 or 14, wherein the openings or holes and the potted piece are potted with polyurethane.

15. The apparatus of any of the examples 12-14, wherein:
the adhesive comprises a silicone coating chemically bonding the electrode cables to the first layer.

16. The apparatus of any of the examples 12-15, wherein:
the second layer extends beyond the first layer so as to form a lip, wherein the adhesive is on the lip and in a region between the first layer and the second layer so as to form a seal waterproofing the electrode cables.

17. The apparatus of any of the examples 12-16, wherein the first layer and the second layer form a skin.

18. The apparatus of any of the examples 12-17, wherein:
the cables are chemically bonded to the adhesive and the potting piece,
each of the cables include a silicone insulated wire joined to a polyurethane insulated wire,
the silicone insulated wire is chemically bonded to the adhesive,
the polyurethane insulated wire is potted in potting material comprising resin in the potted piece and the polyurethane insulated wire is chemically bonded to the resin via marine grade heat shrink, and
the cables exiting potted piece at an end of the potted piece opposite the second layer are bundled and surrounded by a shield comprising steel, heat shrink, electrical tape, and a nylon sheath.

19. The apparatus of example 18, further comprising a splice joint between the silicone insulated wire and the polyurethane insulated wire, wherein the joint is covered in heat shrink and an epoxy coating chemically bonded to the resin.

20. The apparatus of any of the examples 12-19, wherein:
an external surface of the potted piece comprises a plurality of 3D printed plastic ridges (e.g., 1 mm high by 2 mm wide) and the ridges are bonded to the first layer using epoxy (e.g., Gflex epoxy) so as to waterproof the electrode cables by preventing water intrusion into the potted piece, and
an outer surface of the second layer comprises a plurality of 3D printed plastic ridges (e.g., 1 mm high by 2 mm wide) and the ridges are bonded to a skin of the animal using epoxy (e.g., Gflex epoxy).

21. The system or apparatus of any of the examples 12-20, comprising a plurality of data channels, wherein each of the channels is transmitted along two different ones of the electrode cables for redundancy in case one of the electrode cables transmitting the each of the data channels is damaged or disconnected.

22. The apparatus of any of the examples 12-21, comprising a headcap for an animal or human.

23. The apparatus of any of the examples 12-22, wherein the electrode cables connect the electrodes to the data logging device of any of the examples 1-11.

24. The apparatus or system of any of the examples 1-23, wherein the electrode cables transmit biometric data or electrophysiological data (e.g., EEG, EOG, EMG, and/or ECG data) sensed by the electrodes attached to the animal and the data logging device records the data.

25. The apparatus of system of any of the examples, wherein the potted piece or 3D mold resist at least 200 kg of water pressure.

26. The apparatus or system of any of the examples, wherein the electrodes comprise or consist essentially of gold and/or brass.

### BRIEF DESCRIPTION OF THE SEVERAL VIEWS OF THE DRAWINGS

FIG. 1. A history of electrophysiological sleep recordings in marine mammals, including non-invasive and wild recordings in other mammals and birds, and wild electrophysiological recordings of marine mammals (ECG only) to highlight the shifts in recording methodologies over time and across vertebrate systems.

FIGS. 2A-2C. Wire assembly, attachment, and headcap cross section. (FIG. 2A) Wiring schematic from electrodes to the 21 pin underwater connector with callouts showing wire shielding and sheathing, as well as the internal structure of the plotted splice joint between electrode and connector wires. (FIG. 2B) Diagram showing attachment placement and method for each component including logger, ECG, EMG, and headcap patches. (FIG. 2C) Headcap cross section showing internal components of the headcap including the splice joints within the 3D-printed mold, and each successive layer of shielding and sheathing until the outer layer of nylon mesh.

FIGS. 3A-3E. (FIG. 3A) Exploded view of logger housing demonstrating each component of the encapsulation including the housing, grooves for zipties, spacers, battery, mounting tray, screws, support ring, O-rings, Ortman key, housing cap, and cap components (pressure sensor, acrylic window, spacers, retaining rings, and SUBCONN® 21-pin Micro Circular connector. (FIG. 3B) Inset to show the custom printed circuit board (PCB) which holds the logger and routes 21 signals to connectors on its underside to connect signals to the waterproof 21-pin connector. (FIGS. 3C & 3D) Top and side views of the logger demonstrating the position of the logger when mounted on the custom PCB. FIG. 3E. View of housing cap.

FIG. 4. Table 1. Description of animals involved in this study, denoting animal ID, recording location (TMMC: The Marine Mammal Center, LML: Long Marine Lab), date of recording (between 2 May 2019 and 24 Oct. 2020), age class (weanling (post-nursing): 1-6 months, yearling: 6-12 months, juvenile: 1-3 years old), age (in months), sex (determined visually as male [M] or female [F]), standard length (cm), axillary girth (cm), mass (in kilograms), drug(s) administered (TEL: Telazol® [tiletamine and zolazepam], Euthasol® [pentobarbital sodium and phenytoin sodium], KET: ketamine, and VAL: valium), health status (reason for euthanasia or release- all rehabilitated animals were suffering from malnutrition), EEG recording device (stationary amplifier or portable EEG datalogger), type of recording (during euthanasia or release exam procedure or a deployment in captivity), and the total recording duration in hours.

FIGS. 5A-5I—Sleep categorization methods. Sleep stages were distinguished from distinct characteristics of the EEG spectrogram, z-axis gyroscope (for breath detection), and. heart rate. Spectral power varied across stages from (FIG. 5A) slow (10 s) oscillations between slow waves and waking during Drowsiness (DW); FIG. 5B highest amplitude low-frequency activity during SWS (exemplified by hot colors in low frequencies [0.5-4 Hz] of the spectrogram); FIG. 5C lowest amplitude high-frequency activity during REM (exemplified by dark colors in the spectrogram), and FIG. 5D low-amplitude high-frequency activity during quiet waking (QW), and FIG. 5E motion artifacts during active waking (AW). We differentiated between periods of REM with FIG. 5F low heart rate variability (HRV) and FIG. 5G high HRV (independent of changes in respiratory state—apnea [not breathing] and eupnea [breathing consistently]). We demonstrate HRV patterns due to FIG. 5G respiration. FIG. 5H independent of respiration, and FIG. 5I due to both respiration and movement artifacts (due to short-duration inaccuracies in automated peak detection). During active waking, motion artifacts could be caused by large breaths or active forward movement (‘galumphing’ on land or swimming in water).

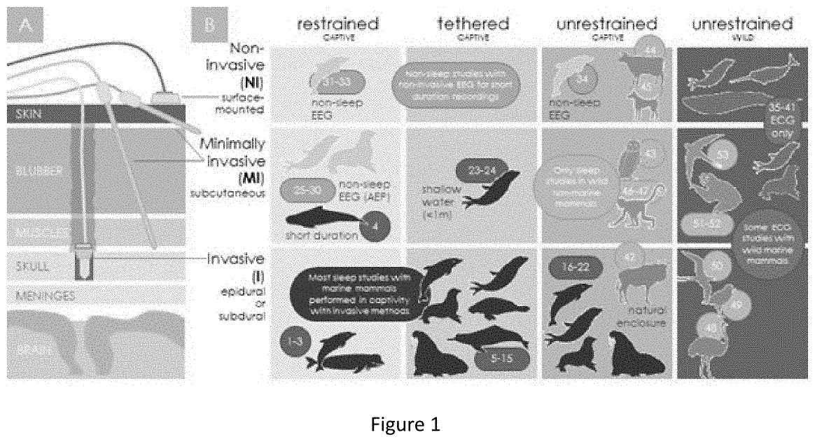

### DETAILED DESCRIPTION

In the following description of the preferred embodiment, reference is made to the accompanying drawings which form a part hereof, and in which is shown by way of illustration a specific embodiment in which the invention may be practiced. It is to be understood that other embodiments may be utilized and structural changes may be made without departing from the scope of the present invention.

### Technical Description

The present disclosure describes a biometric data logging system for an animal, the system comprising (a) an electrophysiological data logging device; (b) a connector configured to route signals to the electrophysiological data logging device; (c) a housing containing the electrophysiological data logging device, wherein the housing is configured to accept a plurality of electrode cables that connect to and/or plug into the connector, and the housing comprises a lightweight and pressure resistant wall surrounding the electrophysiological data logging device and a first acrylic window within the wall configured to allow visualization of the electrophysiological data logging device; and a flexible nylon mesh attached to the housing and configured to affix the housing to an outside of the animal.

### I. Example Instrumentation Electrode Configurations and Attachment

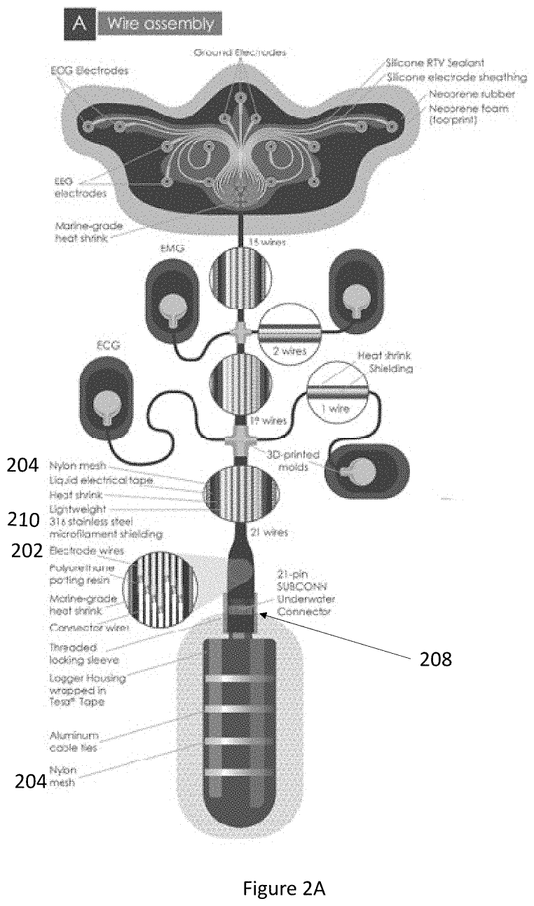

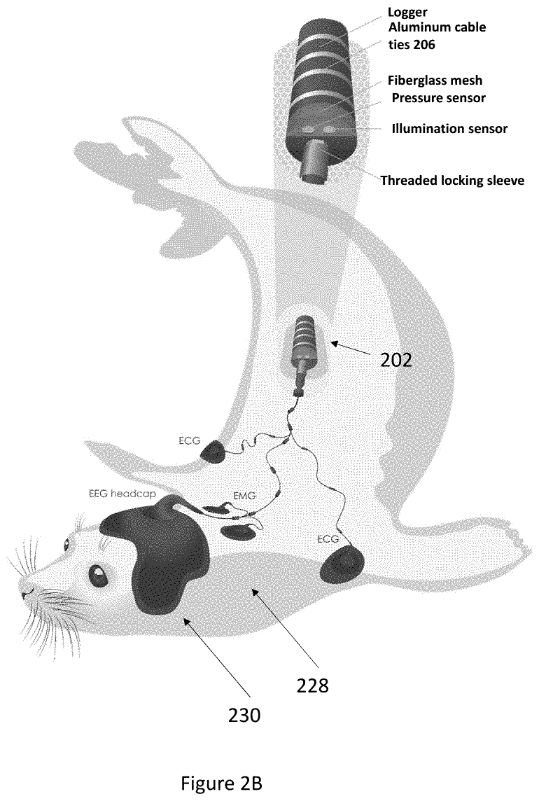

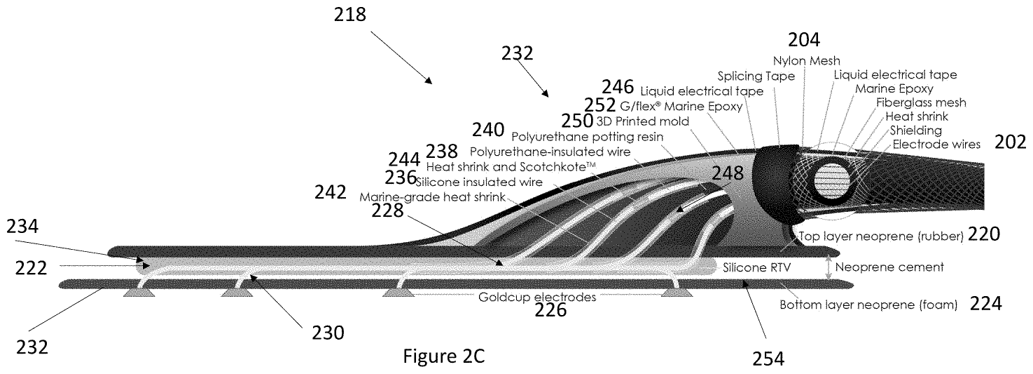

Non-invasive, surface-mounted Genuine Grass® goldcup electrodes were used to measure 9 differential electrophysiological channels (4 electroencephalogram [EEG], 2 electrooculogram [EOG], 2 electromyogram [EMG], and 1 electrocardiogram [ECG]). EEG electrodes were placed over the frontal and parietal derivations of each hemisphere, EOG electrodes were placed approximately 5 centimeters posterior to the outer canthus, EMG electrodes were placed above the nuchal muscles, ECG electrodes were placed on either side of the body near the fore flippers, and ground electrodes were placed on the forehead between the supraorbital vibrissae (FIG. 2). This electrode configuration closely matches montages used for implanted polysomnography in other pinnipeds (Kendall-Bar et al., 2019; Lyamin et al., 2012; Lyamin, Lapierre, et al., 2008; Lyamin, Mukhametov, et al., 2002; Mukhametov et al., 1985). Recordings were performed with anesthetized seals at The Marine Mammal Center (TMMC) where the sedated animal was tethered to a stationary amplifier (PowerLab™) (Seals #1-11), while a portable EEG datalogger was used to perform recordings from freely moving seals at Long Marine Lab (Seals #12-16) (Table 1). In both cases, the ultimate electrode configuration was similar, although multiple electrode types and configurations were tested during anesthesia procedures using the live-feed stationary amplifier to optimize signal detection. These optimal electrode types and configurations are discussed in the following sections.

Electrodes were attached to clean skin using conductive paste and kinesiology tape during stationary EEG recordings with anesthetized animals at The Marine Mammal Center. For recordings with the portable EEG datalogger on animals instrumented at Long Marine Lab, waterproofed electrodes were attached by embedding into flexible neoprene patches. Prior to instrumentation, animals were anesthetized with Telazol® (˜1 mg/kg). The use of sedative agents was not necessary to alleviate pain but was performed in order to ensure the safety of personnel during handling of a wild animal. The fur was trimmed and the skin was cleaned with alcohol or acetone at electrode attachment sites to ensure adequate signal conduction and adhesive efficacy. These electrodes were attached to clean skin with flexible neoprene adhesive (Aquaseal™) EEG, EOG, and ground electrodes were embedded into a single EEG headcap, and EMG and ECG patches were attached separately to each side of the body (FIG. 2).

### Headcap and Patch

FIG. 2 illustrates a design for the EEG headcap and patches, wherein the electrodes are embedded between two layers of neoprene. In one embodiment, the electrodes are embedded between two layers of neoprene foam, while other embodiments combined an outer layer of durable, flexible neoprene rubber (3 mm thick, 40 A durometer) with a second, inner layer of thin neoprene foam (3 mm thick) to hold electrodes in place against the skin. This design facilitates reuse of the equipment by preventing tearing in the upper neoprene layer. Between the two layers of neoprene, the electrodes were fixed into place using a silicone RTV adhesive (Permatex® Adhesive Sealant) to protect the wires and facilitate maintenance of wire configuration across deployments. The sealant created a strong mechanical bond to abraded neoprene rubber, a chemical bond to silicone-insulated electrode sheathing, and no bond to the neoprene cement (added after the full cure time of the silicone RTV) that peeled off easily upon each retrieval of the instrument.

Several precautions were taken, where each electrode exited the upper layer of neoprene rubber, to minimize water intrusion. In one embodiment, a sealant (e.g., neoprene cement) comprising a waterproof seal was applied between the two layers of neoprene foam. The wire output was lifted from the patch to avoid interrupting the seal of each patch's footprint and minimize water intrusion.

Holes were created in the upper neoprene rubber layer through which each wire could exit and these wires were routed through custom-designed 3D-printed molds. After creating these 3D-molds, an extra layer of neoprene foam was inserted on either side of the mold was needed to closely conform to the curve of the head.

A chemical bond to the electrodes' silicone insulation was achieved using the Permatex® Adhesive Sealant mentioned above.

Electrode wires were tested for material compatibility. The Technomed goldcup electrodes were insulated with a type of thermoplastic polyurethane (TPU) that created a chemical bond with two-part Epoxies® urethane resin potting compound (Part No.: 20-2180). Each silicone-insulated Genuine Grass electrode was spliced to a Technomed electrode's TPU wire to achieve a chemical bond between the electrode wire and potting compound at several points along the wire. Water intrusion to this splice joint was prevented by implementing several sequential layers: a) normal heat shrink, b) ScotchKote™, c) marine-grade heat shrink, d) ScotchKote™, e) marine-grade heat shrink, f) urethane potting compound, and g) silicone RTV Permatex® Adhesive Sealant (only on the silicone-insulated inner end). The abraded marine-grade heat shrink created a chemical bond with the urethane potting compound and the silicone RTV created a chemical bond with the silicone insulation and held the wires in place. This process was repeated for each patch, using smaller 3D molds for the 1- and 2-wire outputs from EMG and ECG patches.

The first iteration included copper braided shielding surrounding each cable bundle followed by an outer layer of heat shrink. In some places, repairs to the cable were required where the shielding had pierced the heat shrink using liquid electrical tape. Although the copper braided shielding began to corrode over the course of the 5-day experiment, this first iteration yielded cleaner, higher quality signals than the second iteration, where unshielded Teflon® wires coated in liquid electrical tape or loosely bundled with nylon braided sheathing were used. This was especially true for signals such as heart rate, which rely on relatively long wires going down the side of the animal and was less problematic for the bundle of 15 cables from the headcap. For the final iteration, cable bundles were surrounded with ultra-lightweight braided microfilament 316L stainless steel shielding material, heat shrink, liquid electrical tape, and nylon braided sheathing (FIG. 2).

### Underwater Connector

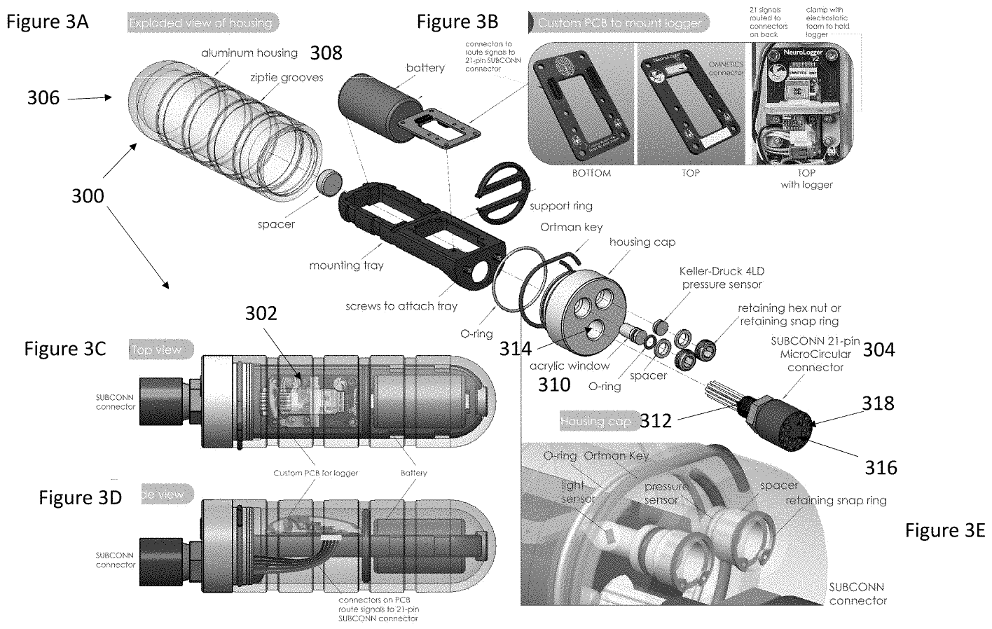

FIGS. 2 and 3 illustrate electrode cables were soldered to the leads of a 21-pin underwater connector which routed the electrical signals into the portable datalogging unit. These solder joints were staggered to maximize space-efficiency and strength, covered in heat-shrink, and potted into the urethane potting compound mentioned above and covered with large-diameter marine-grade heat shrink to minimize water intrusion at mechanically vulnerable connections. A small lip on either side of the underwater connector was used to retain the threaded locking sleeve that reduces the possibility of the connector becoming disconnected during the experiment.

### Datalogger Housing

FIG. 3 illustrates a portable, waterproofed, and ruggedized housing for an electrophysiological datalogging acquisition device “Neurologger 3®” (©2016 Evolocus LLC) that incorporated signals from various sensors (up to 18 differential electrophysiological channels, inertial motion sensors: 3D accelerometer, 3D gyroscope, 3D magnetic compass, environmental sensors: Keller-Druck 4LD pressure transducer, temperature sensor, and illumination sensor), displayed recording status via a small LED, and transmitted small snapshots of electrophysiological data via Bluetooth.

A housing for the device was used, as opposed to potting the device into epoxy resin, in case a datalogger repair or adjustment was needed. The integration of multiple sensors and maintenance of logger functionality and flexibility for this housing was non-trivial given multiple concerns: (1) the illumination sensor must be placed against a transparent housing wall, (2) the pressure sensor must be precisely machined between an internal lip and external retaining wall, (3) maintenance of Bluetooth capabilities (signals are attenuated by aluminum housing material), and (4) integration of 21 electrophysiological signals (each of which is a potential conduit for water intrusion).

The use of robust high-density polycarbonate (a type of bulletproof glass) was investigated because of its relative lightness, ability to transmit Bluetooth, monitor the status LED, and check for water intrusion. A robust SubConn® Micro Circular 21-pin underwater connector was selected for the transmission of electrophysiological signals into the housing. This housing failed a pressure test at ˜1900 psi but was used for the first sleep recording in the captive environment. For the next design, the polycarbonate walls were replaced with thinner aluminum walls and inserted 2 acrylic windows where the illumination sensor was located, the LED status could be visualized, and Bluetooth signals could penetrate to allow us to verify signal quality. This housing passed pressure testing to 3000 psi before it was used for deployments with wild animals. This design was refined for further studies, creating a cylindrical housing with one semi-spherical end and a threaded cap at the other side that integrates pressure and illumination sensors as well as the SubConn® Micro Circular 21-pin underwater connector (FIG. 3).

The datalogger was attached to the fur with epoxy via a flexible nylon mesh, consistent with established best practices for external attachment of animal telemetry tags (Horning et al. 2019) (FIG. 2). When time allowed, datalogger signal quality was verified by examining 20-second Bluetooth snapshots of the raw signals.

### II. Example Studies and Characterization of the System

Understanding of electrophysiological sleep in marine mammals has been limited by the lack of non-invasive methods and the absence of studies in the wild, where natural sleep patterns can be observed within the ecological context in which they evolved. Throughout the animal kingdom, there has been a long-standing call for electrophysiological sleep studies in the natural environment (Allison, 1972), and researchers are beginning to take advantage of technological advances, which have permitted recordings of electrophysiological sleep in sloths, frigatebirds, and barn owls in the field (Allison, 1972; Rattenborg et al., 2008, 2017).

The study presented herein addresses this call by providing substantial advances along multiple dimensions to allow the classification of sleep in wild marine mammals: (1) the validation of non-invasive surface mounted electrodes to record sleep over multiple days on land and in water, (2) the creation of a portable, robust, and pressure-proofed device to maximize animal mobility, and (3) the application of sophisticated signal processing techniques to maximize signal quality. A systematic framework, which capitalizes on technological advances to facilitate future sleep studies on wild marine mammals, is provided.

### Example 1—Validation of Surface-Mounted Electrodes

Hans Berger performed the first non-invasive scalp EEG recording on a human shortly after the first invasive EEG recording in 1924 (Berger, 1929; İnce et al., 2020). In addition, surface mounted disk electrodes were validated during early marine mammal auditory evoked response studies, by comparison to brain implants (Bibikov, 1992). The work disclosed herein collected side-by-side recordings from needle and surface electrodes during an auditory evoked response to compare the amplitude of brain signal detection using each electrode type. Previous comparisons of needle and surface electrodes for humans and animal applications have demonstrated that both methods provide reliable recordings across the EEG frequency spectrum, despite minimal differences in contaminating artifacts (Scriba et al., 2013; Zablow & Goldensohn, 1969).

### Example 2—Animal Mobility: Allowing Free Range of Motion, in the Wild

In addition to introducing a non-invasive method for signal detection, the study presented herein allowed the animal free range of motion in the wild. Many previous marine mammal sleep studies required some form of physical restraint, such as stretchers (very little motion possible) or harnesses (50 cm in any direction (Lyamin, Shpak, et al., 2002)). Other studies included animals with skull implants or needle electrodes who were “tethered” to a stationary amplifier via cables (e.g. 6-meter-long artifact-free cables in the case of several dolphin studies (Mukhametov et al., 1976) and 12 northern elephant seal pups (Castellini et al., 1994; Milsom et al., 1996)). Several marine mammal sleep studies involving implanted electrodes provided greater animal mobility using telemetered devices that allowed the animals to freely swim and dive within captive pools (Table 1: (Lyamin et al., 2012; Lyamin, Lapierre, et al., 2008; Ridgway et al., 1975)).

To date, studies that allowed free range of motion have always employed invasive methods. Such invasive methods have a greater potential for infection or injury, and are therefore not suitable for use outside of captivity where continuous visual observation of the animal is not possible. In addition, telemetered devices designed for free-swimming animals in captivity are not suitable for use in the wild in deep diving marine mammals where they would encounter higher pressures. The disclosed invention therefore provides a rugged, portable recording platform which, unlike previous devices, is designed to withstand mechanical stresses from massive (˜200 kg) animals resting on and scraping the device against rocks as well as any water pressure that a wild northern elephant seal might encounter during diving activity (up to 3000 psi).

### Example 3—Signal Processing: Maximizing Signal Quality using Independent Component Analysis

Surface recordings are preferable from an animal welfare perspective because they reduce the risk of infection by maintaining the skin's protective barrier; however, they can introduce signal quality challenges. The thickness and resistance of each intermediate tissue can reduce the amplitude, accuracy, and precision of EEG measurements 8-fold from the scalp versus implantation on the skull in humans (Buzsáki et al., 2012; Wendel et al., 2010). Lower amplitude signals are overcome more easily by exogenous and endogenous electrical artifacts (Klemm, 1965). Improvements in data acquisition systems and signal processing algorithms have advanced our ability to identify, filter out, and remove contaminating signals. These algorithms, such as Independent Components Analysis (ICA), show promise for automating artifact identification and removal in human neuroscience studies (Onton & Makeig, 2006; Romero et al., 2003). ICA has also been applied to improve signal detection using surface-mounted suction cup electrodes in dolphin auditory evoked potential recording. This study validates the efficacy and utility of using ICA to identify and remove orthogonal components that contain electrical artifacts.

Technological miniaturization, materials innovations, and advances in signal processing technology have supported non-invasive recordings of EEG in freely moving barn owls, piglets, and dairy cows (de Camp et al., 2018; Scriba et al., 2013; Ternman et al., 2012), sleep studies in the wild (using invasive methods (Rattenborg et al., 2008, 2016)), and electrocardiogram in free-ranging marine mammals in the wild (Goldbogen et al., 2019; Horning et al., 2019; Williams et al., 2017). Our study adds to this body of work by providing a waterproof, pressure-resistant platform and rigorous signal processing techniques that allows non-invasive electrophysiological sleep recordings in wild elephant seals.

### Example 4—Materials and Methods

All animal procedures were approved at the federal and institutional levels under National Marine Fisheries Permits #19108, #23188, and #18786 (TMMC), and by the Institutional Animal Care and Use Committee (IACUC) of University of California Santa Cruz (Costd1709 and Costd2009-2) and The Marine Mammal Center (TMMC #2019-2).

This study was designed to validate the use of non-invasive surface mounted goldcup EEG electrodes for reliable sleep detection in wild seals on land and in water over multiple days. First, stationary recordings were performed with a live-feed EEG amplifier to refine electrode configuration and methods at The Marine Mammal Center with anesthetized animals (N=11; Seals #1-11). Next, five captive deployments of a portable EEG datalogger were performed to establish behavioral and electrophysiological correlates of sleep using non-invasive methods on freely moving animals in a controlled lab environment at Long Marine Lab (N=5; Seals #12-16).

### (i) Adhesive and Electrode Configuration Testing using Stationary Recordings at The Marine Mammal Center (TMMC)

Adhesives were tested for safety and efficacy with recently deceased northern elephant seal specimens at The Marine Mammal Center. The electrode configuration was refined during stationary EEG recordings with 11 anesthetized (Telazol® [tiletamine and zolazepam] used for induction and maintenance) northern elephant seal weanlings (weaned pups ˜3-4 months old) undergoing rehabilitation at The Marine Mammal Center (Table 1). Each recording lasted less than an hour and coincided with routine veterinary procedures.

### (ii) Testing using Captive Deployments—Long Marine Lab (LML)

Once the techniques to attach and configure electrodes were established, multiple day sleep recordings were performed with a portable EEG datalogger in the controlled lab environment to closely monitor the impact of the instrument on the seal, continuously monitor and thoroughly document its behavior, and to establish a baseline of sleep recording quality on land and in water using this surface-mounted recording method. Five juvenile seals (2 yearlings [˜8 months old] and 3 juveniles [˜1 year and 8 months old]) were temporarily translocated from Año Nuevo State Park to the Long Marine Lab marine mammal facility on the Coastal Science Campus at the University of California, Santa Cruz, where the seals were instrumented (Table 1).

Following initial chemical immobilization (1 mg/kg Telazol® [tiletamine and zolazepam]), juvenile seals were transported 21 miles south to the Long Marine Lab, using identical translocation procedures as many previous studies with elephant seals (Mitani et al., 2009; Oliver et al., 1998). The animals were checked on for regular heart rate and breathing at multiple stops and, when conditions dictated, were wetted down to avoid overheating. Shortly after transport, seals were anesthetized (initial dose: 1 mg/kg Telazol® [tiletamine and zolazepam], maintained with ketamine and valium) and instrumented with the portable EEG datalogger (attachment procedure described later in detail under “Instrumentation”).

Seals were then given access to a dry enclosure measuring 20′ by 10′ (6.1 m by 3.0 m) for at least 48 hours after sedation, including a 24-hour recovery period and another 24-hour period during which the anesthetic agents were no longer impacting the animal's physiology. After this recording period on land, the animals were introduced to another enclosure with a pool measuring 16′ by 10′ by 4.5′ deep (4.9 m by 3.0 m by 1.4 m—pool volume 21 m³) and haul-out area measuring 4′ by 10′ (1.2 m by 3.0 m). After an acclimatization period that ranged from 30 minutes to 50 hours, the seals learned to exit the pool and freely transition between the two media. Reattachment procedures were always performed at least 24 hours after the attachment sedation and prior to the final removal sedation to allow time for recovery. A final sedation was performed at the end of the recording, when instrumentation was removed and the seal was rolled back into the aluminum transport cage for transfer back to Año Nuevo State Park, where the seal exited the cage and rejoined the colony.

### (iii) Data Processing

Electrophysiological data was collected from the animals included in this study, some of which were collected from anesthetized animals at The Marine Mammal Center and some were collected from free-moving animals at Long Marine Lab.

Electrophysiological data from stationary recordings at The Marine Mammal Center was collected and analyzed in LabChart® (ADlnstruments). The remainder of this section refers to data processing for the 7 recordings on freely moving animals at Long Marine Lab.

Electrophysiological data were sampled at 500 Hz and inertial motion and environmental sensors were sampled at approximately 36 Hz. This binary data was stored on a 256 GB microSD card, processed using a custom MATLAB script (Neurologger Converter & Visualizer © Evolocus LLC), and then converted into .MAT (MATLAB file) and .EDF (European Data Format) formats.

When animals entered the water, ECG artifacts were sometimes present in EEG channels, complicating visual and quantitative scoring methods. We processed sections of this data collected in water (where ECG artifacts were present) to remove ECG artifacts and enable visual and quantitative EEG scoring. We applied the “runica” Independent Components Analysis function in the open source EEGLAB toolbox in MATLAB. Independent Component Analysis (ICA) is now standard practice in EEG signal processing that refers to a collection of unsupervised learning algorithms that decompose multivariate data into maximally independent components and (Romero et al., 2003). The ICA algorithm was trained with a subset of the data collected from animals while they were stationary underwater. The ICA weights were visualized and if the heart rate decomposed separately from EEG signals into at most 2 or 3 channels, these ICA weights were selected and applied across the larger dataset. Intact ECG channels (without the ICA ECG decompositions removed) were always maintained for subsequent heart rate analysis.

Inertial motion sensor data was calibrated and processed using the Customized Animal Tracking Systems (CATS) toolbox to obtain measures of ODBA (overall dynamic body acceleration), pitch, roll, and heading (Cade et al., 2016; Johnson, 2011). During calibration and processing, the data sampled at 250/7 Hz were down sampled to a standard integer frequency (25 Hz).

The electrophysiological signals were examined alongside processed 3-axis accelerometer data in LabChart (©ADInstruments). Instantaneous heart rate was calculated using the cyclic measurement peak detection algorithm in LabChart® with the ECG peak detection parameters consistent with those used for large mammals, exceeding a minimum of 2 standard deviations with a QRS width of 60 milliseconds across normalized 4-second windows. Heart Rate Variability (HRV) was calculated using LabChart® to generate statistics such as low frequency power, which is critical in the analysis of heart rate variation during potential periods of paradoxical sleep.

### (iv) Qualitative Sleep Analysis

To prepare the raw data for visual sleep scoring, electrophysiological signals were bandpass filtered according to the standard outlined in the American Academy of Sleep Medicine (AASM) sleep scoring manual: EEG/EOG: 0.3-30 Hz; EMG: 10-100 Hz; ECG: 0.3-75 Hz. All signals were visualized using standard temporal and voltage scales (100 μV for EEG/EOG, 40 μV for EMG, 2 mV for ECG, [−1.5,1.5] G-forces (g) for accelerometer data). Power spectra were visualized for two (L & R) of the best EEG channels using Fast Fourier Transform (FFT) using a Hann (cosine-bell) data window with sample size of 1024 points and 50% overlap, displaying Power Density from 0-15 μV²/Hz for frequencies between 0-15 Hz.

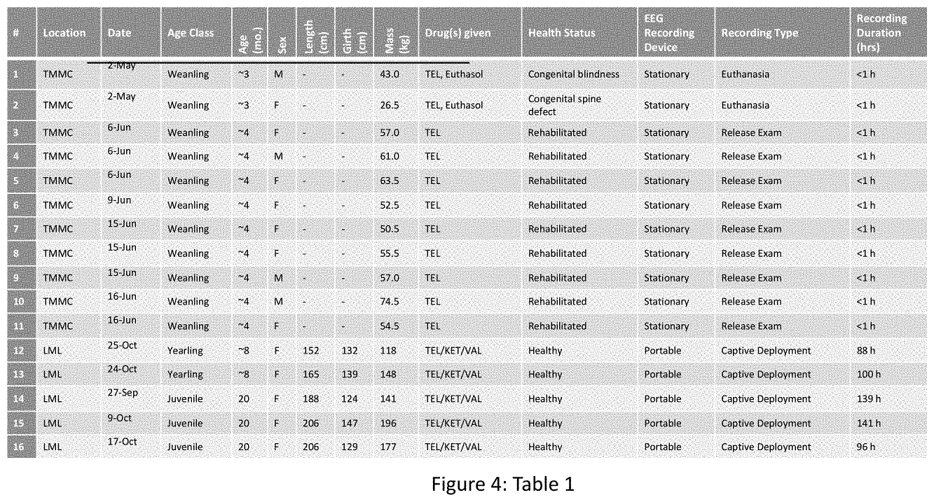

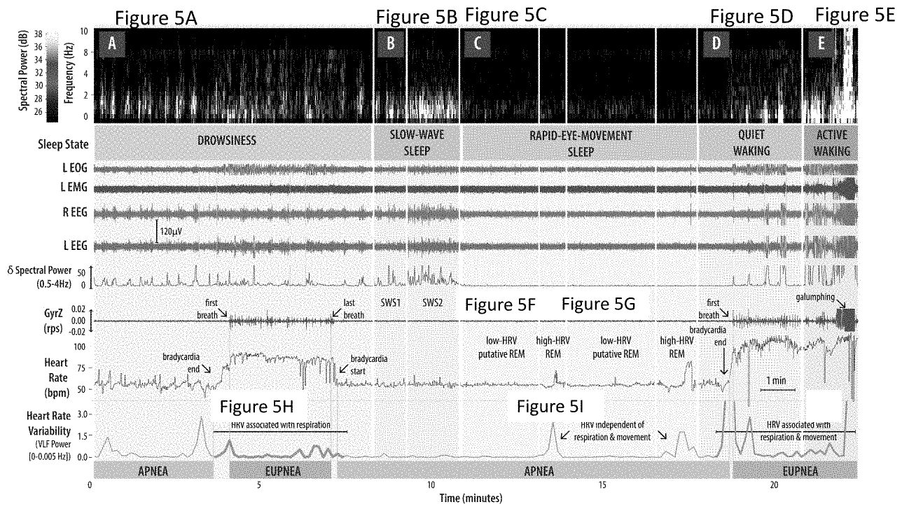

Guidelines for visual scoring were based off those set for other marine mammals (Castellini et al., 1994; Kendall-Bar et al., 2019; Lyamin, 1993; Lyamin et al., 2012; Milsom et al., 1996; Ridgway et al., 1975). Previous studies of sleep in northern elephant seals utilized purely visual sleep scoring methods (Castellini et al., 1994). Given the new application of surface-mounted electrodes to detect sleep in marine mammals, we scored sleep stage transitions instantaneously and precisely within 5 seconds to maximize the precision of resulting quantitative metrics to train our sleep scoring automation algorithm. We marked transitions between the following states: (1) Sleep Stages: Active Waking (AW), Quiet Waking (QW), Light Sleep (LS), Slow Wave Sleep (SWS), Paradoxical Sleep (PS); (2) Bradycardia versus Tachycardia (based on heart rate); (3) Eupnea versus Apnea (based on fine-scale spikes present in accelerometer signals) (FIG. 5).

Accelerometer data (derived from the attachment location on the back of the animal) provided an accurate proxy for respiration, where each breath was visible with small spikes (peak-to-peak amplitude of ˜0.1 g [G-forces]) amid background low amplitude oscillations (<0.05 g) (FIG. 5). During the majority of sleep cycles, the animal's first and last breaths were clearly visible in the accelerometer data. When there was too much noise in the accelerometer data to pick out individual breaths, breaths were marked within 5 seconds of the start or end of a bradycardia.

Heart rate patterns were scored separately from breaths because the anticipatory increase in heart rate often occurred several (˜10 seconds) before the first breath, which skewed heart rate metrics for that state. Likewise, heart rate often hovered near tachycardic rates for many seconds after the last detected breath before descending into the apneic bradycardia. This scoring paradigm allows us to examine heart rate patterns with respect to sleep stages while accounting for the heart rate fluctuations associated with respiratory events.

Active Waking (AW) was characterized whenever significant motion artifacts (longer duration or greater amplitude than eye blinks) were present in electrophysiological channels and was confirmed by visual examination of accelerometer traces. Quiet Waking (QW) was characterized by low voltage, high frequency background EEG activity, occasional eye blink artifacts, and accelerometer traces demonstrating only breathing or subtle motion (i.e. slow rolling, grooming, or body repositioning).

A sleep stage previously undescribed in elephant seals, which we termed Light Sleep (LS), was characterized by intermittent high amplitude activity between 1 and 7 Hz (with highest activity between 1-5 Hz) amid a background of low voltage, high frequency waking EEG activity. This stage often followed quiet waking and preceded slow wave sleep. Where high resolution video was available, this stage coincided with eye closure and could occur directly after motion, suggesting that this activity could occur as soon as the eyes were closed (perhaps similar to alpha rhythms in humans).

A transition to Slow Wave Sleep (SWS) was characterized by the appearance of continuous high amplitude (>10 μV²/Hz) delta power between 0.5 and 4 Hz lasting longer than 10 seconds. The peak-to-peak amplitude of slow waves varied slightly between recordings and recording location (land v. water). On land it typically exceeded the standard 75 μV threshold for humans and in water it was slightly lower. In all cases, delta spectral power was ˜5× greater during sleep than during neighboring periods of quiet waking or potential paradoxical sleep. Slow wave sleep could follow a period of quiet waking or light sleep if light sleep was present. Slow wave sleep occurred independently of breathing and always involved nearly symmetrical high amplitude activity in all four EEG channels, very low muscle activity in the EMG channels, and no visible eye activity in the EOG channels (besides some low frequency EEG contamination).

Rapid-eye movement (REM sleep) was characterized by very low voltage EEG activity and greater low frequency heart rate variability, often directly following slow wave sleep (delta spectral power ˜5× lower than the amplitude during preceding slow wave sleep). We conservatively scored REM sleep only when both low voltage activity and heart rate variability were observed. Heart rate variability in this state was large (sometimes from as high as 82 to as low as 20 bpm in a single apnea) and much slower than in other states (such as SWS, LS, and QW), which often had stepwise arrythmias consisting of a few beats per minute. Occasionally, eye movements (detected in only EOG channels) and whisker twitches occurred in this stage. Muscle activity (measured with EMG on the neck) was sometimes lower than during SWS, but usually remained unchanged if EMG activity was already low in SWS. Occasionally, large whole body muscle jerks would occur in this stage.

### (v) Quantitative Sleep Analysis

Statistics were generated for the duration of each sleep stage in LabChart® (sleep stage duration, start and end times, seconds elapsed, and mean and standard deviation for: heart rate and R-R interval for ECG, integral of EMG activity, delta spectral power (0.5-4 Hz), theta spectral power (4-8 Hz), alpha spectral power (8-13 Hz), and beta spectral power (>13 Hz) for all four EEG channels). Spectral power analyses were performed with a Fast Fourier Transform (FFT) using a Hann (cosine-bell) data window with sample size of 1024 points and 50% window overlap. We also generated additional statistics related to heart rate variability (HRV). Among HRV statistics, we found Low Frequency power (between 0.05-0.1 Hz) to be most helpful in discriminating the slow oscillations in heart rate during REM sleep from the shorter, stepwise arrhythmias during other stages of sleep and wakefulness. We split each sleep stage into 10 second epochs which were analyzed in R (R Core Team, 2020) and visualized using ggplot2 (Wickham, 2009).

First, 10 second epochs for each sleep stage were analyzed independently of changes in breathing and heart rate. Then, sleep stages were separated into four substages, coinciding with: periods of tachycardia, periods of bradycardia, periods of transition from bradycardia to tachycardia (from bradycardic heart rate to first breath detected via accelerometer), and vice versa (from last breath detected via accelerometer to start of bradycardic heart rate).

### Device Embodiments

A device according to embodiments described herein can be embodied in many ways including, but not limited to, the following.

1. FIGS. 2 and 3 illustrates an example water resistant biometric data logging system 300 for an animal 302, the system comprising:
(a) an electrophysiological data logging device 302;
   (b) an underwater connector 304 configured to route signals to the electrophysiological data logging device;
(c) a housing 306 containing the electrophysiological data logging device, wherein:
   the housing is configured to accept a plurality of electrode cables 202 connected to the underwater connector, and
   the housing comprises a metal (e.g., aluminum) wall 308 (lightweight and strong for pressure, high density polycarbonate glass failed) surrounding the electrophysiological data logging device and a first acrylic window 310 within the first aluminum wall configured to allow visualization of the electrophysiological data logging device; and
a flexible nylon mesh 204 attached to the housing and configured to affix the housing to an outside of the animal.

2. The system of example 1, further comprising a plug 312 comprising a screw configured to be screwed into a screw hole 314 in the housing, wherein the plug includes an opening 316 receiving the underwater connector so as to operably connect the underwater connector to the housing and the data logging device.

3. The system of example 1, wherein:
the system further comprises zip ties 206 attaching the housing the mesh and epoxy for gluing the mesh to the animal, and
the housing comprises a cylinder or a capsule.

4. The system of example 1 wherein the electrode cables are soldered to the underwater connector, thereby forming a plurality solder joints 208, and wherein each of the solder joints are potted into a flexible epoxy splice joint.

5. The system of any of examples 1 or 4, wherein the underwater connector comprises more than 10 pin connectors 318 or at least 21 pin connectors, wherein each of the pin connectors comprises a solder joint 208 soldered to a different one of the electrode cables and each of the solder joints is potted in the underwater connector for waterproofing.

6. The system of examples 4 or 5 wherein each of the solder joints is covered in marine grade heat-shrink.

7. The system of any of the examples 1-6, wherein each of the electrode cables comprises aerospace grade electrical shielding 210 with 3/16 stainless steel around a wire.

8. The system of any of the examples 1-7 comprising at least 5, 10, 15, 20, 21, or 25 of the electrode cables 202.

9. The system of example 1 wherein one or more of the electrode cables terminate in a headcap 218, the headcap comprising a first neoprene layer 220, a mold 222 configured to accept the plurality of the electrode cables beneath the first neoprene layer; and a second neoprene layer 224 between the mold and the first neoprene layer configured to hold the electrodes 226 in place against the skin 228 of the animal 230.

10. The system of example 1 wherein at least one of the plurality of electrode cables comprises electrical shielding 210 around the at least one of the electrode cables.

11. The system of example 10 where the electrical shielding 210 comprises braided copper or aluminum foil.

12. FIG. 2 illustrates an apparatus 218 for mounting electrodes 226 onto a body (e.g., of an animal 230 or human), comprising:
a first layer 220 comprising a first plurality of openings 228 or holes, the first layer comprising or consisting essentially of synthetic rubber;
a second layer 224 comprising a second plurality of openings 230 or holes, the second layer comprising a foam or a sacrificial material;
a potted piece 232 containing a plurality of electrode cables 202, wherein:
   the electrode cables are routed through the first plurality of openings and the second plurality of openings or holes, and
   the first layer is between the potted piece and the second layer;
a plurality of electrodes 226 terminating the electrode cables, wherein the electrodes are configured to be mounted (e.g., adhered to) a surface of the body; and
an adhesive 222 between the first layer and the second layer, wherein the adhesive adheres the electrode cables 202 to the second layer.

13. The apparatus of example 12, wherein the first layer and the second layer comprise or consist essentially of neoprene.

14. The apparatus of example 13 or 14, wherein the openings or holes and the potted piece are potted with polyurethane.

15. The apparatus of any of the examples 12-14, wherein:
the adhesive 222 comprises a silicone coating chemically bonding the electrode cables to the first layer.

16. The apparatus of any of the examples 12-15, wherein:
the second layer extends beyond the first layer so as to form a lip 232, wherein the adhesive is on the lip and in a region between the first layer and the second layer so as to form a seal 234 waterproofing the electrode cables.

17. The apparatus of any of the examples 12-16, wherein the first layer and the second layer form a skin.

18. The apparatus of any of the examples 12-17, wherein:
the cables are chemically bonded to the adhesive and the potting piece,
each of the cables include a silicone insulated wire 236 joined to a polyurethane insulated wire 238,
the silicone insulated wire is chemically bonded to the adhesive,
the polyurethane insulated wire is potted in potting material 240 comprising resin in the potted piece and the polyurethane insulated wire is chemically bonded to the resin via marine grade heat shrink 242, and
the cables exiting potted piece at an end of the potted piece opposite the first layer are bundled and surrounded by a shield comprising steel 210, heat shrink 244, electrical tape 246, and a nylon sheath 204.

19. The apparatus of example 18, further comprising a splice joint 248 between the silicone insulated wire and the polyurethane insulated wire, wherein the joint is covered in heat shrink and an epoxy coating chemically bonded to the resin.

20. The apparatus of any of the examples 12-19, wherein:
an external surface of the potted piece comprises a plurality of 3D printed plastic ridges 250 (e.g., 1 mm high by 2 mm wide) and the ridges are bonded to the first layer using epoxy (e.g., Gflex epoxy 252) so as to waterproof the electrode cables by preventing water intrusion into the potted piece.

21. The system or apparatus of any of the examples 12-20, comprising a plurality of data channels, wherein each of the channels is transmitted along two different ones of the electrode cables 202 for redundancy in case one of the electrode cables transmitting the each of the data channels is damaged or disconnected.

22. The apparatus of any of the examples 12-21, comprising a headcap for an animal or human.

23. The apparatus of any of the examples 12-22, wherein the electrode cables connect the electrodes 206 to the data logging device 302 of any of the examples 1-11.

24. The apparatus or system of any of the examples 1-23, wherein the electrode cables transmit biometric data or electrophysiological data (e.g., EEG, EOG, EMG, and/or ECG data) sensed by the electrodes attached to the animal and the data logging device records the data.

25. The apparatus of system of any of the examples, wherein the potted piece or 3D mold resist at least 200 kg of water pressure.

26. The apparatus or system of any of the examples, wherein the electrodes comprise or consist essentially of gold and/or brass.

### Advantages and Improvements

The studies evidence the first non-invasive recordings of marine mammal sleep and the first recordings of marine mammal sleep in the wild. The novel approach to waterproof electrodes allows non-invasive and maintains the protective barrier of the skin (while all previous studies use invasive methods which penetrate the skin and/or skull). In addition, the animal-borne device is pressure-proofed to 3000 psi (over a mile deep) and incorporates over 20 electrophysiological signals, more than have been previously integrated into an animal biotelemetry tag.

The invention and validation provide substantial advances along multiple dimensions to allow the classification of sleep in wild marine mammals: (1) the validation of non-invasive surface mounted electrodes to record sleep over multiple days on land and in water, (2) the creation of a portable, robust and pressure-proofed device to maximize animal mobility, by allowing free-range of motion in the wild, and (3) the application of sophisticated signal processing techniques to maximize signal quality. By integrating multiple individual methodological advances, a systematic framework is provided which capitalizes on technological advances to facilitate future sleep studies on marine mammals

Because this approach is non-invasive, it does not leave a conduit vulnerable to infection as do invasive methods which either penetrate the surface of the skin or involve drilling holes into the skull. Of course, a non-invasive approach is also preferable from an ethical and animal welfare perspective. The robust, pressure-proofed housing allows us to record sleep in naturally behaving animals within the natural context in which sleep evolved, instead of within the confines of an artificial captive environment. Furthermore, by integrating state of the art signal processing techniques, the yield of quantitative data from our non-invasive electrodes can be maximized while applying rigorous statistical treatment and computational analyses. The application of Independent Components Analysis (a now-standard tool for EEG signal processing in humans) is novel given that almost no marine mammal sleep studies have applied signal processing techniques beyond simple filtering of the data or manual artifact removal.

### Conclusion

This concludes the description of the preferred embodiment of the present invention. The foregoing description of one or more embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

What is claimed is:

1. A water resistant biometric data logging system for an animal, the system comprising:
(a) an electrophysiological data logging device;
(b) an underwater connector configured to route signals to the electrophysiological data logging device;
(c) a housing containing the electrophysiological data logging device, wherein:
the housing is configured to accept a plurality of electrode cables that connect to and/or plug into the underwater connector, and
the housing comprises an aluminum wall surrounding the electrophysiological data logging device and a first acrylic window within the aluminum wall configured to allow visualization of the electrophysiological data logging device; and
a flexible nylon mesh attached to the housing and configured to affix the housing to an outside of the animal.

2. The system of claim 1, further comprising a plug comprising a screw configured to be screwed into a screw hole in the housing, wherein the plug includes an opening receiving the underwater connector so as to operably connect the underwater connector to the housing and the data logging device.

3. The system of claim 1, wherein:
the system further comprises zip ties attaching the housing the flexible nylon mesh and epoxy for gluing the mesh to the animal, and
the housing comprises a cylinder or a capsule.

4. The system of claim 1 wherein the electrode cables are soldered to the underwater connector, thereby forming a plurality solder joints, and wherein each of the solder joints are potted into a flexible epoxy splice joint.

5. The system of claim 1, wherein:
the underwater connector comprises more than 10 pin connectors,
each of the pin connectors comprises a solder joint soldered to a different one of the electrode cables, and
each of the solder joints is potted in the underwater connector for waterproofing.

6. The system of claim 5 wherein each of the solder joints is covered in marine grade heat-shrink.

7. The system of claim 1, wherein each of the electrode cables comprises aerospace grade electrical shielding with 3/16 stainless steel around a wire.

8. The system of claim 1, comprising at least 5, 10, 15, 20, 21, or 25 of the electrode cables.

9. The system of claim 1 wherein one or more of the electrode cables terminate in a headcap, the headcap comprising:
a first neoprene layer,
a mold configured to accept the plurality of electrode cables beneath the first neoprene layer; and
a second neoprene layer between the mold and the first neoprene layer configured to hold the electrodes in place against the skin of the animal.

10. The system of claim 1 wherein at least one of the plurality of electrode cables comprises electrical shielding around the at least one of the electrode cables.

11. The system of claim 10 where the electrical shielding comprises braided copper or aluminum foil.

12. An apparatus for mounting electrodes onto a body, comprising:
a first layer comprising a first plurality of openings, the first layer comprising or consisting essentially of synthetic rubber;
a second layer comprising a second plurality of openings, the second layer comprising a foam;
a potted piece containing a plurality of electrode cables, wherein:
the electrode cables are routed through the first plurality of openings and the second plurality of openings or holes, and
the first layer is between the potted piece and the second layer;
a plurality of electrodes terminating the electrode cables, wherein the electrodes are configured to be mounted on a surface of the body; and
an adhesive between the first layer and the second layer, wherein the adhesive adheres the electrode cables to the second layer.

13. The apparatus of claim 12, wherein the first layer and the second layer comprise or consist essentially of neoprene and the openings and the potted piece are potted with polyurethane.

14. The apparatus of claim 12, wherein:
the adhesive comprises a silicone coating chemically bonding the electrode cables to the second layer.

15. The apparatus of claim 12, wherein:
the second layer extends beyond the second layer so as to form a lip, wherein the adhesive is on the lip and in a region between the first layer and the second layer so as to form a seal waterproofing the electrode cables.

16. The apparatus of any of the claim 12, wherein the first layer and the second layer form a skin.

17. The apparatus of claim 12, wherein:
the electrode cables are chemically bonded to the adhesive and the potting piece,
each of the electrode cables include a silicone insulated wire joined to a polyurethane insulated wire,
the silicone insulated wire is chemically bonded to the adhesive,
the polyurethane insulated wire is potted in potting material comprising resin in the potted piece and the polyurethane insulated wire is chemically bonded to the resin via marine grade heat shrink, and
the electrode cables exiting the potted piece at an end of the potted piece opposite the first layer are bundled and surrounded by a shield comprising steel, heat shrink, electrical tape, and a nylon sheath.

18. The apparatus of claim 17, further comprising a splice joint between the silicone insulated wire and the polyurethane insulated wire, wherein the splice joint is covered in heat shrink and an epoxy coating chemically bonded to the resin.

19. The apparatus of claim 12, wherein:
an external surface of the potted piece comprises a first plurality of 3D printed plastic ridges and the first plurality of 3D printed ridges are bonded to the first layer using epoxy so as to waterproof the electrode cables by preventing water intrusion into the potted piece.

20. A data logging system comprising the apparatus of claim 12, further comprising:
a data logging device connected to the electrodes via the electrode cables, the electrode cables transmitting each of a plurality of data channels along two different ones of the electrode cables for redundancy in case one of the electrode cables transmitting the each of the data channels is damaged or disconnected; and wherein:
the data channels transmit biometric data or electrophysiological data sensed by the electrodes attached to the body comprising an animal; and
the data logging device is configured to record the biometric data.
